(19) United States
(12) Patent Application Publication
(10) Pub. No.: US 2005/0209653 A1
(43) Pub. Date: Sep. 22, 2005

(54) Intra-luminal device for gastrointestinal electrical stimulation

(75) Inventors: Timothy P. Herbert, Maple Grove, MN (US); Warren L. Starkebaum, Plymouth, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/801,230

(22) Filed: Mar. 16, 2004

(51) Int. Cl. A61N 1/18

(52) U.S. Cl. 607/40; 607/133

(57) ABSTRACT

An intra-luminal device for gastrointestinal electrical stimulation is self-powered and self-contained within a capsule-like housing, and is capable of non-surgical implantation within the patient. The device includes an implantable pulse generator and one or more electrodes mounted within a common device housing. The device housing is capable of endoscopic introduction to a desired location within the gastrointestinal tract, such as the stomach, via the esophagus. The device may be appropriate for short-term, mid-term or trial stimulation applications.

### FIELD OF THE INVENTION

The invention relates to medical devices for maintaining gastrointestinal health and, more particularly, medical devices for electrical stimulation of the gastrointestinal tract.

### BACKGROUND

Gastroparesis is an adverse medical condition in which normal gastric motor function is impaired. Gastroparesis results in delayed gastric emptying as the stomach takes too long to empty its contents. Typically, gastroparesis results when muscles within the stomach or intestines are not working normally, and movement of food through the stomach slows or stops. Patients with gastroparesis typically exhibit symptoms of nausea and vomiting, as well as gastric discomfort such as bloating or a premature or extended sensation of fullness, i.e., satiety. The symptoms of gastroparesis are the result of reduced gastric motility. Gastroparesis generally causes reduced food intake and subsequent weight loss, and can adversely affect patient health.

Electrical stimulation of the gastrointestinal tract has been used to treat symptoms of gastroparesis. For example, electrical stimulation of the gastrointestinal tract, and especially the stomach, is effective in suppressing symptoms of nausea and vomiting secondary to diabetic or idiopathic gastroparesis. Typically, electrical stimulation involves the use of electrodes implanted in the muscle wall of the target organ, e.g., the muscle wall of the stomach in the case of gastric stimulation. The electrodes are electrically coupled to an implanted or external pulse generator via implanted or percutaneous leads. The pulse generator delivers a stimulation waveform via the leads and electrodes. An example of an implanted pulse generator suitable for gastric stimulation is the ITREL 3, commercially available from Medtronic, Inc., of Minneapolis, Minn. Gastric stimulation devices work well to suppress symptoms associated with gastroparesis. However, gastric stimulation devices typically require surgical implantation of both the electrodes, leads and typically the pulse generator. Although surgical implantation may be appropriate for long-term electrical stimulation, some patients may experience symptoms for a relatively brief period of time, i.e., a few weeks or less. For example, some patients may experience symptoms similar to gastroparesis for a short time. For example, patients may experience nausea and vomiting for a short time following surgery. In these cases, however, it may not be desirable to subject the patient to the risk of surgery. Instead, non-surgical techniques for deployment of the stimulation electrodes and pulse generator are desirable.

U.S. Pat. No. 3,411,507 to Wingrove describes a temporary stimulation system to treat post-operative ileus. The system described in this patent includes a portable, external stimulator carried outside the body. The stimulator is attached to a temporarily implanted electrode via a naso-gastric tube that is placed in the stomach. A ground pad is provided to serve as the indifferent electrode.

Goyal et al. describe another temporary stimulation system in the article entitled “Gastrointestinal electrical stimulation (GES) can be performed safely with endoscopically placed electrodes,” Amit Goyal, Sandeep Khurana, Sandeep Bhragava, Abell L. Thomas, American Journal of Gastroenterology 96 (9), 2001. In the Goyal et al. system, temporary screw-in cardiac stimulation electrodes are inserted through an endoscope and screwed into the mucosa of the stomach. Leads extend from the electrodes to an external pulse generator via the patient's mouth.

The systems described by the Wingrove and Goyal et al. permit stimulation to be delivered on a temporary basis and avoid the need for surgery. However, the Wingrove and Goyal et al. systems require external wires that pass through the patient's mouth or nose in order to connect the pulse generator to the electrode. Persistent trans-nasal or trans-oral access can be uncomfortable for the patient and increases the risk of dislodgement of the electrode placed in the interior of the stomach.

Table 1 below lists examples of documents, including the Wingrove patent and Goyal et al. article, that disclose techniques for electric stimulation of the gastrointestinal tract to alleviate symptoms of nausea and vomiting, including symptoms caused by gastroparesis or post-operative ileus.

TABLE 1

| Document | Inventors/Authors | Title |
|---|---|---|
| U.S. Pat. No. | Wingrove | Method of Gastrointestinal |
| 3,411,507 | | Stimulation with Electrical |
| | | Pulses |
| American Journal | Goyal et al. | Gastrointestinal electrical |
| of Gastroenterology | | stimulation (GES) can be |
| 96 (9), 2001 | | performed safely with |
| | | endoscopically placed electrodes |
| U.S. Pat. No. | Mintchev et al. | Gastro-intestinal Electrical |
| 6,243,607 | | Pacemaker |
| U.S. Pat. No. | Chen et al. | Gastro-intestinal pacemaker |
| 5,690,691 | | having phased multi-point |
| | | stimulation |
| U.S. Pat. No. | Bourgeois | Method and apparatus for |
| 6,216,039 | | treating irregular gastric rhythms |
| U.S. Pat. Pub. No. | Swoyer et al. | Implantable medical device |
| 20020103424 | | affixed internally within the |
| | | gastrointestinal tract |
| U.S. Pat. No. | Jenkins | Gastric stimulator apparatus and |
| 6,606,523 | | method for installing |

All documents listed in Table 1 above are hereby incorporated by reference herein in their respective entireties. As those of ordinary skill in the art will appreciate readily upon reading the Summary of the Invention, Detailed Description of the Preferred Embodiments and Claims set forth below, many of the devices and methods disclosed in the patents of Table 1 may be modified advantageously by using the techniques of the present invention.

### SUMMARY OF THE INVENTION

In general, the invention is directed to techniques for electrical stimulation of the gastrointestinal tract using an intra-luminal device that is capable of non-surgical implantation within the patient. The device includes an implantable pulse generator and one or more electrodes within a common device housing. The device housing may be capsule-like and capable of endoscopic introduction to a desired location within the gastrointestinal tract, such as the stomach, via the esophagus. In addition, the device is self-contained and includes no external components that would require persistent trans-oral or trans-nasal access to the device. The device may be particularly appropriate for short-term, mid-term or trial stimulation applications.

Various embodiments of the present invention provide solutions to one or more problems existing in the prior art with respect to prior devices for gastrointestinal electrical stimulation. These problems include the inability of existing electrical stimulation devices to be implanted without surgery. Conversely, many existing electrical stimulation devices designed for chronic implantation are not readily removable, and may require surgical procedures for explant. As a further problem, the few existing stimulation devices that do not require surgical implantation still involve persistent passage of electrical leads through a patient's nose or mouth, creating discomfort to the patient and increasing the possibility that electrodes may be dislodged. As a result of the combination of problems above, electrical stimulation devices have not been widely used for patients requiring only short-term stimulation, such as patients who experience symptoms of nausea or vomiting, e.g., due to post-operative ileus or following chemotherapy.

Various embodiments of the present invention are capable of solving at least one of the foregoing problems. When embodied in a device for gastrointestinal electrical stimulation, for example, the invention includes various features that facilitate the delivery of gastrointestinal electrical stimulation on a short-term or trial basis without the need for surgical implantation or explantation techniques. In addition, the device may be endoscopically positioned at a desired location within the gastrointestinal tract without surgery, and without the protrusion of leads or other components from the patient's nose or mouth. The device may be securely fixed within a body lumen, and reduce the possibility that electrodes may become dislodged from a target position for delivery of electrical stimulation. In addition, in some embodiments, the device requires no explant procedure. Rather, the device can be made to self detach from the gastrointestinal tract wall for passage through the patient's body. Accordingly, the device may eliminate one or more of the problems that have limited the short-term use of gastrointestinal electrical stimulation to alleviate symptoms such as nausea and vomiting.

Various embodiments of the invention may possess one or more features to solve the aforementioned problems in the existing art. In some embodiments, a stimulation device according to the invention includes a device housing sized for introduction into a gastrointestinal tract. An electrical pulse generator is mounted within the device housing, and generates an electrical stimulation waveform. One or more electrodes are electrically coupled to the electrical pulse generator and mounted to the device housing to deliver the electrical stimulation waveform to the gastrointestinal tract. A fixation structure attaches the device housing to a surface within the gastrointestinal tract.

The stimulation device may take the form of a capsule-like member that combines the pulse generator, electrodes, and fixation structure within a common device. The capsule may include any of a variety of fixation structures for attaching the capsule to tissue within the gastrointestinal tract, such as the mucosal lining of the esophagus or stomach. In some embodiments, the stimulation device may be delivered by an endoscopic delivery device that includes a handle and a flexible probe that extends from the handle into the gastrointestinal tract of the patient. In such embodiments, the capsule is coupled to a distal end of the probe for delivery to a particular location within the gastrointestinal tract.

In comparison to known techniques for electrical stimulation of the gastrointestinal tract, various embodiments of the invention may provide one or more advantages. For example, a stimulation device in accordance with the invention can be deployed within the patient without the need for surgical procedures. Rather, the device can be endoscopically placed at a location within the gastrointestinal track via the patient's nose or mouth. The pulse generator and electrodes can be mounted within a common device housing, such as a capsule. Therefore, in addition to avoiding surgery, there is no need for leads to extend from the patient's nose of mouth. On the contrary, the entire device is contained within the gastrointestinal tract and includes a fixation structure to attach the device directly to tissue within the gastrointestinal tract. Consequently, a device in accordance with the invention eliminates the need for surgery and reduces patient discomfort. In addition, the device may be readily implanted for short-term treatment, offering a more convenient therapy for patients suffering from symptoms such as nausea or vomiting following surgery or chemotherapy. The device also may be suitable for trial stimulation to predict the efficacy of chronic implantation of a gastrointestinal stimulation device for a given patient. As a further advantage, the stimulation device may even be used as a preventative treatment for nausea or vomiting, thereby reducing in-house medical expenses associated with treatment of such symptoms. Also, in some embodiments, the device may be self-detachable, endoscopically detachable or possibly endoscopically retrievable, requiring no surgical procedure for explant.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating a gastrointestinal electrical stimulation system shown in conjunction with a patient.

FIG. 2 is a functional block diagram illustrating a gastrointestinal electrical stimulation device.

FIG. 3 is a schematic diagram illustrating deployment of the device of FIG. 2 within a patient's gastrointestinal tract.

FIG. 4 is a cross-sectional side view illustrating positioning of a stimulation device within the gastrointestinal tract with a tissue fixation structure using a vacuum cavity and pin.

FIG. 5 is a cross-sectional side view of the device of FIG. 4 with the tissue securing pin advanced through tissue within the vacuum cavity.

FIG. 6 is a cross-sectional side view of the device of FIG. 5 following removal of an endoscopic delivery device.

FIG. 7 is a side view of a stimulation device within the gastrointestinal tract with a tissue fixation structure using a pair of barbed hooks.

FIG. 8 is a side view of a stimulation device within the gastrointestinal tract with an alternative tissue fixation structure using a pair of barbed hooks.

FIG. 9 is a cross-sectional side view illustrating exemplary arrangement of internal components of the stimulation device shown in FIG. 4.

FIG. 10 is a bottom plan view of the stimulation device of FIG. 9 with a vacuum cavity and tissue securing pin.

FIG. 11 is a bottom plan view of an alternative stimulation device with a vacuum cavity and a pair of tissue securing pins.

FIG. 12 is a cross-sectional side view of a stimulation device with a fixation structure that combines barbed hooks with a vacuum cavity.

FIG. 13 is a cross-sectional side view of a stimulation device with a fixation structure that combines a barbed hook with a pair of vacuum cavities.

FIG. 14 is a side view of a stimulation device with a fixation structure in the form of an expandable frame.

FIG. 15 is a cross-sectional view of the device and expandable frame of FIG. 14 in an unexpanded state within a body lumen.

FIG. 16 is a cross-sectional view of the device and expandable frame of FIG. 14 in an expanded state within a body lumen.

FIG. 17 is cross-sectional side view of another stimulation device with a capsule-like structure and a screw-like fixation structure.

FIG. 18 is a top view of the device of FIG. 17.

FIG. 19 is a cross-sectional side view of the device of FIG. 17 with an endoscopic positioning probe.

FIG. 20 is a schematic diagram illustrating insertion of a stylet into the mucosal lining of the stomach.

FIG. 21 is a schematic diagram illustrating introduction of fluid through the stylet of FIG. 20 to create an expanded implant pocket.

FIG. 22 is a schematic diagram illustrating implantation of the device of FIG. 17 into the implant pocket shown in FIG. 21.

FIG. 23 is a timing diagram illustrating various parameters of an electrical stimulation waveform for gastrointestinal stimulation.

FIG. 24 is a flow diagram illustrating implantation and operation of a gastrointestinal electrical stimulator.

### DESCRIPTION OF THE PREFERRED EMBODIMENTS

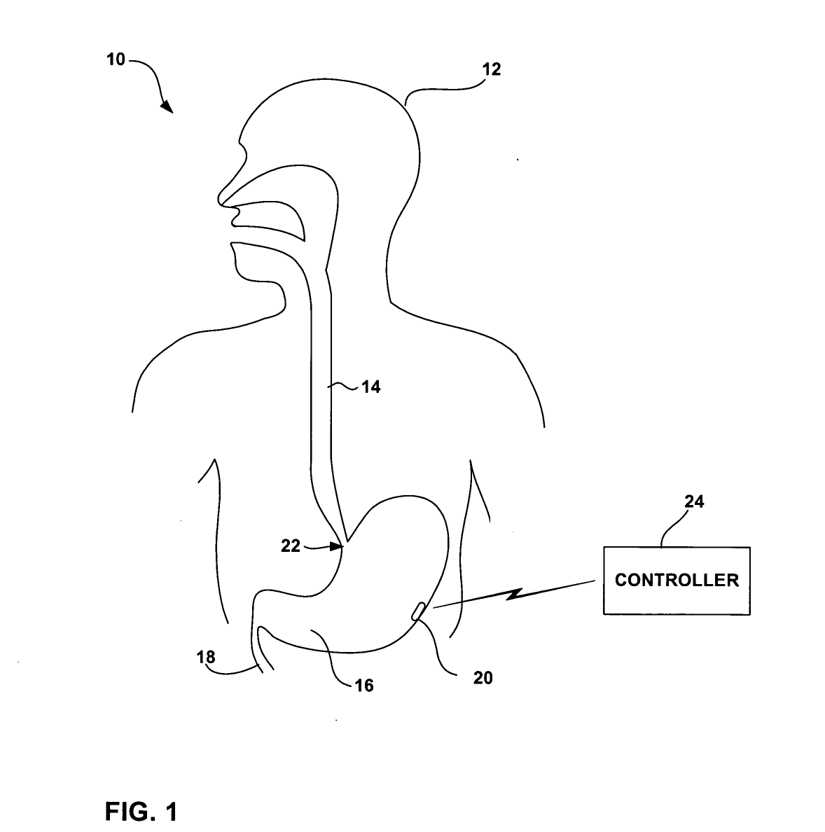

FIG. 1 is a schematic diagram illustrating a gastrointestinal electrical stimulation system 10 shown in conjunction with a patient 12. In the illustrated embodiment, stimulation system 10 delivers electrical stimulation to a target location within the gastrointestinal tract, such as the esophagus 14, stomach 16, small intestine 18, or colon (not shown). Stimulation system 10 includes a stimulation device 20, which may be placed at a target location by endoscopic delivery. In particular, stimulation device 20 may be delivered via the oral or nasal passage of patient 12 using an endoscopic delivery device. In the example of FIG. 1, stimulation device 20 resides within stomach 16. In this case, the endoscopic delivery device traverses esophagus 14 and then enters into stomach 16 via lower esophageal sphincter 22 of patient 12.

Stimulation device 20 may have a capsule-like device housing sized for endoscopic introduction via esophagus 14 and, in some embodiments, passage through the gastrointestinal tract. For example, the capsule-like device housing of stimulation device 20 have a maximum length of less than approximately 10 mm and a maximum width of less than approximately 5 mm. In some embodiments, the device housing may be substantially cylindrical, in which case the housing may have a maximum height of less than approximately 10 mm and a maximum diameter of less than approximately 5 mm. The capsule-like device housing of stimulation device 20 includes a power source, a pulse generator, one or more electrodes, and a fixation structure. The pulse generator produces an electrical stimulation waveform with parameters selected to suppress symptoms such as nausea and vomiting. The fixation structure secures stimulation device 20 to a target location within the gastrointestinal tract. In particular, fixation structure may perforate the mucosa and lodge in the muscularis extema of the gastrointestinal tract wall when introduced against the mucosa, or grip a fold of the mucosa. The electrodes are thereby placed in contact with tissue at the target location to deliver the electrical stimulation waveform to patient 12. The capsule-like device housing may be substantially cylindrical, with a length greater than its diameter and flat or rounded ends, although the invention is not limited to any particular shape.

To place stimulation device 20, a distal end of the endoscopic delivery device is inserted into esophagus 14 and guided to a target location within the gastrointestinal tract. Following placement of stimulation device 20, the endoscopic delivery device is withdrawn from patient 12 once the stimulation device is attached to a target site. Hence, surgery is not required to place stimulation device 20 within patient 12. Moreover, following placement of stimulation device 20, there are no leads or other connections that extend outside of patient 12. On the contrary, stimulation device 20 is entirely self-contained, self-powered and integrated within a common, capsule-like housing.

Stimulation device 20 may be used to treat disorders such as nausea or vomiting or dysmotility disorders that ordinarily would require surgical implantation of an electrical stimulation system or one or more leads that extend outside the patient's body. The endoscopically placed stimulation device 20 can be used to treat short-term disorders of a few days to a few weeks, or even mid-term disorders from a few weeks to a year or more, without the need for surgery or external wires. In light of the convenience of stimulation device 20, it may even be used as a preventative treatment for nausea or vomiting associated with gastrointestinal surgery, general surgery, chemotherapy, functional dyspepsia, pregnancy, or other similar procedures known to have secondary responses such as nausea or vomiting.

The fixation structure may take any of a variety of forms, such as one or more pins, hooks, barbs, screws, sutures, clips, pincers, staples, tacks, or other fasteners. In some embodiments, the fixation structure can at least partially penetrate the mucosal lining of the gastrointestinal tract. In other embodiments, the fixation structure may be an expandable frame, such as a stent, that carries stimulation device 20. Examples of suitable biocompatible materials for fabrication of the fixation structure include stainless steel, titanium, polyethylene, nylon, PTFE, nitinol, or the like.

Other examples include surgical adhesives that supplement the attachment made by the fixation structure or serve as the fixation structure itself. In other words, a pin, hook or other fixation structure may be accompanied by a biocompatible, surgical adhesive, or the adhesive may be used as the sole fixation structure without mechanical fasteners. Hence, the adhesive may work alone or in combination with a mechanical fastener.

Examples of suitable surgical adhesives for bonding the stimulation device to the mucosal lining include any of a variety of cyanoacrylates, derivatives of cyanoacrylates, or any other adhesive compound with acceptable toxicity to human gastrointestinal cells that provides the necessary adhesion properties required to secure the stimulation device 20 to the target location for a period of time sufficient for delivery of electrical stimulation. Adhesives may be injected or otherwise applied into the region surrounding the target location, e.g., via a channel within the endoscopic delivery device, or carried by the stimulation device 20 itself.

Stimulation device 20 may be configured to eventually self-detach from the target location. For example, stimulation device 20 may detach from the mucosal lining of esophagus 14 or stomach 16, when a portion of the lining held by the fixation structure sloughs away. In this case, the stimulation device 20 is free to pass through the gastrointestinal tract for excretion by the patient 12. Typically, it may be desirable that the fixation structure is effective for a period of at least a few days, and possibly up to several weeks, so that there is adequate time for delivery of electrical stimulation to treat the patient's symptoms. Alternatively, in some embodiments, stimulation device 20 may be detached by applying pressure from an endoscopic tool, or by introducing an endoscopic tool to actively cut the attachment structure and permit the stimulation device to pass through the gastrointestinal tract. In other embodiments, an endoscopic tool may be used to detach stimulation device 20 and retrieve it, i.e., remove it through the oral or nasal passage of patient 12.

In some embodiments, the fixation structure, including pins, expandable frames, and the other structures described above, may be made form a degradable material that degrades or absorbs over time at the attachment site to release stimulation device 20 from tissue at the target location. In either case, upon detachment, stimulation device 20 passes through the gastrointestinal tract of patient 12. U.S. Pat. Nos. 6,285,897 and 6,698,056 to Kilcoyne et al. provide examples of fixation structures for attaching monitoring devices to the lining of the esophagus, including suitable degradable materials. The fixation structures described in the Kilcoyne patents may be suitable for attachment of stimulation device 20. The contents of the Kilcoyne et al. patents are incorporated herein by reference in their entireties.

Examples of suitable degradable materials for fabrication of the fixation structure or structures include bioabsorbable or dissolvable materials such as polylactic acid (PLA) or copolymers of PLA and glycolic acid, or polymers of p-dioxanone and 1,4-dioxepan-2-one, as described in the Kilcoyne patents. A variety of absorbable polyesters of hydroxycarboxylic acids may be used, such as polylactide, polyglycolide, and copolymers of lactide and glycolide, as also described in the Kilcoyne patents.

As further shown in FIG. 1, in some embodiments, stimulation device 20 may communicate with an external controller 24 via wireless telemetry. Controller 24 may permit a user to activate stimulation device 20 and adjust stimulation parameters. For example, a patient 12 or other user may use controller 24 to start stimulation, stop stimulation, set stimulation duration, or adjust stimulation amplitude, frequency, pulse width and duty cycle. Wireless telemetry may be accomplished by radio frequency communication or proximal inductive interaction of controller 24 with stimulation device 20. External controller 24 may take the form of a portable, handheld device, like a pager or cell phone, that can be carried by patient 12.

Controller 24 may include an antenna that is attached to the body of patient 12 at a location proximate to the location of stimulation device 20 to improve wireless communication reliability. Also, in some embodiments, controller 24 may receive operational or status information from stimulation device 20, and may be configured to actively interrogate stimulation device to receive the information.

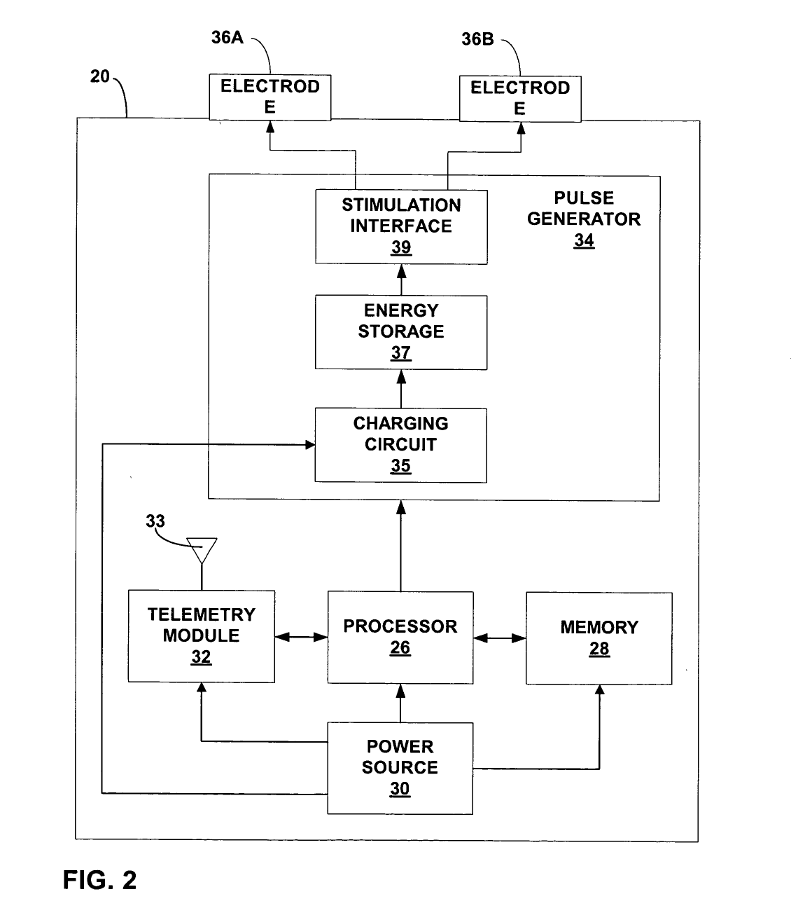

FIG. 2 is a block diagram illustrating exemplary functional components of stimulation device 20. In the example of FIG. 2, stimulation device 20 may include a processor 26, memory 28, power source 30, telemetry module 32, pulse generator 34 and electrodes 36A, 36B. Telemetry module 32 is optional and permits communication with external controller 24 for transfer of data and adjustment of stimulation parameters. Alternatively, in some embodiments, stimulation device 20 may exclude telemetry module 32, in which case all stimulation parameters may be preset and fixed within the stimulation device. Exclusion of telemetry module 32 may be desirable in some applications to achieve reductions in the size of stimulation device 20.

Processor 26 controls operation of stimulation device 20 and may include one or more microprocessors, digital signal processors (DSPs), application-specific integrated circuits (ASICs), field-programmable gate arrays (FPGAs), or other digital logic circuitry. Memory 28 may include any magnetic, electronic, or optical media, such as random access memory (RAM), read-only memory (ROM), electronically-erasable programmable ROM (EEPROM), flash memory, or the like. Memory 28 may store program instructions that, when executed by processor 26, cause the processor to perform the functions ascribed to it herein. For example, memory 28 may store instructions for processor 26 to execute in support of control of telemetry module 32 and pulse generator 34.

Telemetry module 32 may include a transmitter and receiver to permit bi-directional communication between stimulation device 20 and external controller 24. In this manner, external controller 24 may transmit commands to stimulation device 20 and receive status and operational information from the stimulation device. Telemetry module 32 includes an antenna 33, which may take a variety of forms. For example, antenna 33 may be formed by a conductive coil or wire embedded in a housing associated with stimulation device 20. Alternatively, antenna 33 may be mounted on a circuit board carrying other components of stimulation device 20, or take the form of a circuit trace on the circuit board. If stimulation device 20 does not include a telemetry module 32, a magnetic reed switch may be provided in a circuit between power source 30 and the other components of the device so that, with the aid of an external magnet, the device may be turned on at the time the device is placed in the patient. Alternatively, stimulation device 20 may simply be activated upon release from the endoscopic delivery device.

Power source 30 may take the form of a battery and power circuitry. Stimulation device 20 typically may be used for a few days or weeks, and therefore may not require substantial battery resources. Accordingly, the battery within power source 30 may be very small. An example of a suitable battery is a model 317 silver oxide battery often used to power watches. The model 317 battery has voltage of 1.55 volts and a capacity of 12.5 mA-hours and has a disk-like shape with a diameter of approximately 5.7 mm and a thickness of approximately 1.65 mm. With a typical range of power requirements of the stimulation waveform and the components of stimulation device 20, the model 317 battery can be expected to power the device for between approximately two weeks and eighteen months, depending on actual usage conditions.

Different types of batteries or different battery sizes may be used, depending on the requirements of a given application. In further embodiments, power source 30 may be rechargeable via induction or ultrasonic energy transmission, and includes an appropriate circuit for recovering transcutaneously received energy. For example, power source 30 may include a secondary coil and a rectifier circuit for inductive energy transfer. In still other embodiments, power source 30 may not include any storage element, and stimulation device 20 may be fully powered via transcutaneous inductive energy transfer.

Pulse generator 34 produces an electrical stimulation waveform with parameters selected to suppress particular symptoms such as nausea and vomiting. As shown in FIG. 2, pulse generator 34 includes a charging circuit 35, an energy storage device 37, and a stimulation interface 39. Charging circuit 35 converts energy supplied by power source 30 device 37 to charge energy storage device 37, which may be a capacitor. Stimulation interface 39 amplifies and conditions charge from energy storage device 37 to produce an electrical stimulation waveform for application to electrodes 36A, 36B. As an example, pulse generator 34 may incorporate circuitry similar to the pulse generation circuitry in the ITREL 3 neurostimulator, commercially available from Medtronic, Inc. of Minneapolis, Minn.

Stimulation parameters, such as amplitude, frequency, pulse width, duty cycle and duration, may be selected to simply suppress symptoms, or actually treat the cause of the symptoms such as gastroparesis, post-operative ileus or some other disorder that disrupts stomach motility. Stimulation device 20 may be applicable to a variety of disorders, particularly when a small, inexpensive, and temporary device is desired. Hence, processor 26 may be programmed, or pulse generator 34 may be otherwise configured, according to the stimulation requirements of particular disorders. Although stimulation device 20 may be capable of extended or long-term use, temporary use will be described herein for purposes of illustration.

Examples of applications to which stimulation device 20 may be applied include trial screening of gastric electrical stimulation therapy for gastroparesis, or trial screening of gastric electrical stimulation for treatment of obesity, irritable bowel syndrome, functional dyspepsia, and gastroesophageal reflux disease. In these cases, stimulation device 20 may provide a convenient way to evaluate the potential efficacy of gastric electrical stimulation. In particular, with trial stimulation, a physician can determine whether long-term stimulation by surgical implantation of a stimulation device is appropriate for a particular patient. In addition, in some instances, stimulation device 20 may serve as a bridge between short-term relief of nausea and vomiting and the implantation of a long-term solution.

Other example applications include delivery of gastric electrical stimulation for treatment of nausea and/or vomiting resulting from chemotherapy, treatment of post-operative ileus, treatment of hyperemesis gravidarum, and temporary treatment of gastroparesis. Stimulation device 20 may be particularly useful for patients who have acute but severe symptoms but are refractory to drug therapy for such symptoms. Exemplary stimulation parameters for some of the above applications will be described in greater detail below.

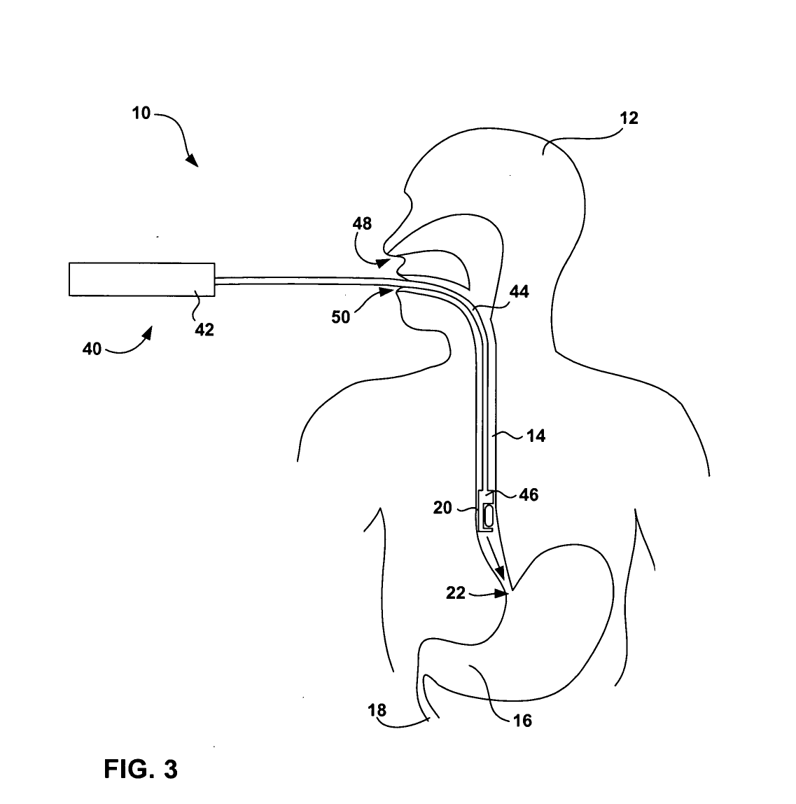

FIG. 3 is a schematic diagram illustrating deployment of stimulation device 20 within the gastrointestinal tract of patient 12. As shown in FIG. 3, an endoscopic delivery device 40 serves to position and place stimulation device 20 within the gastrointestinal tract of patient 12. Delivery device 40 includes a proximal portion, referred to herein as a handle 42, and a flexible probe 44 that extends from handle 42 into the gastrointestinal tract of patient 12. Stimulation device 20 is coupled to a distal end 46 of delivery device 40 for delivery to a target location within the gastrointestinal tract. In the illustrated embodiment, stimulation device 20 is depicted as being in transit to a target location within stomach 16, which is accessed via esophagus 14 and LES 22. Distal end 46 of delivery device 40 enters esophagus 14, via either nasal cavity 48 or oral cavity 50, and extends through esophagus 14 to a desired placement location. Stimulation device 20 is attached to the mucosal lining at a target location within esophagus 14, stomach 16, or small intestine 18, as will be described in greater detail below, and the distal end 46 of delivery device 40 releases stimulation device 20.

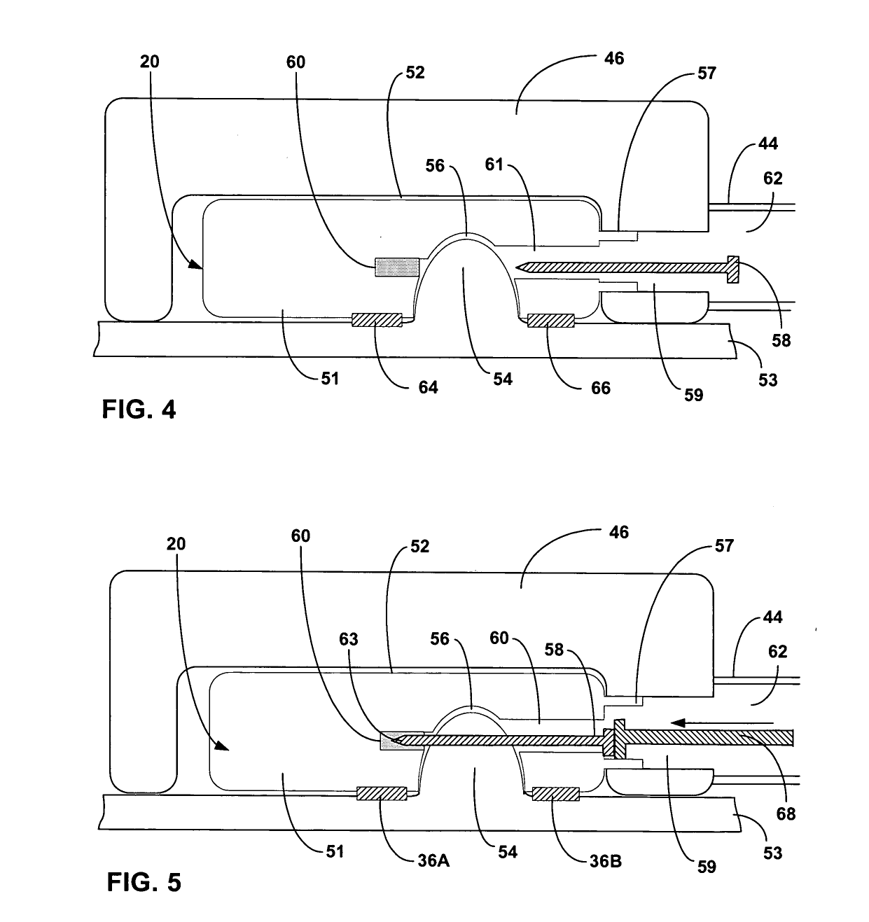

FIG. 4 is a cross-sectional side view illustrating positioning of a stimulation device 20 within the gastrointestinal tract with a fixation mechanism using a vacuum cavity and pin to secure tissue. During placement, stimulation device 20 is held within a placement bay 52 within distal end 46 of endoscopic delivery device 40. As shown in FIG. 4, stimulation device 20 has a capsule-like device housing 51, which may be substantially cylindrical in shape. Device housing 51 may be formed from a variety of biocompatible materials such as stainless steel or titanium. A coupling collar 57 serves to secure a proximal end of device housing 51 within a channel 59 defined by distal end 46 of delivery device 40.

Device housing 51 includes a pulse generator (not shown in FIG. 4), electrodes 36A, 36B, and a fixation structure. Electrodes 36A, 36B are coupled to the pulse generator to deliver stimulation energy to tissue at the target site. A physician guides endoscopic delivery device 40 to place electrodes 36A, 36B in contact with a mucosal lining 53 at the target location of the gastrointestinal tract. Delivery device 40 may include viewing optics to permit the physician to visualize the target location and observe implantation of stimulation device 20. Alternatively, an independent viewing endoscope may be inserted with delivery device 40, or external viewing techniques such as radiography or fluoroscopy may be used.

In the example of FIG. 4, the fixation structure includes a vacuum cavity 56 defined by device housing 51 and a tissue securing pin 58. Upon engagement of stimulation device 20 with mucosal lining 53, the physician activates a vacuum source (not shown) to apply negative pressure to vacuum cavity 56 via a vacuum port 61. The vacuum source is coupled to an internal lumen 62 within flexible probe 44, and is in fluid communication with vacuum port 61. The negative vacuum pressure serves to draw a portion 54 of mucosal lining 53 into vacuum cavity 56. Tissue securing pin 58 is advanced through the tissue 54 held in vacuum cavity 56 to thereby penetrate the tissue 54 and attach device housing 51 to the mucosal lining 53.

The volume of tissue 54 drawn into vacuum cavity 56 and the depth of penetration of pin 58 may be selected to avoid penetration through the wall of the gastrointestinal tract, e.g., the esophageal wall or stomach wall. As an example, it may be desirable to limit the depth of penetration to a range of approximately 1 mm to 15 mm when the site comprises the antrum of the stomach or in the range of approximately 1 mm to 10 mm when the site comprises corpus or fundus to ensure that the fixation structure does not extend substantially through the wall of the gastrointestinal lumen.

FIG. 5 is a cross-sectional side view of the stimulation device 20 of FIG. 4 with the tissue securing pin 58 advanced through tissue within the vacuum cavity 56. As shown in FIG. 5, the physician advances a rod-like member 68 within internal lumen 62 of flexible probe 44 to drive pin 58 into the tissue 54 held in vacuum cavity 56. A distal tip 63 of pin 58 may be received in a bushing 60. Once pin 58 has secured tissue 54, the physician turns off the vacuum source, and releases device housing 51 from placement bay 52 of distal end 46 of delivery device 40. Additional details concerning a similar fixation structure for monitoring devices can be found in the above-referenced Kilcoyne et al. patents.

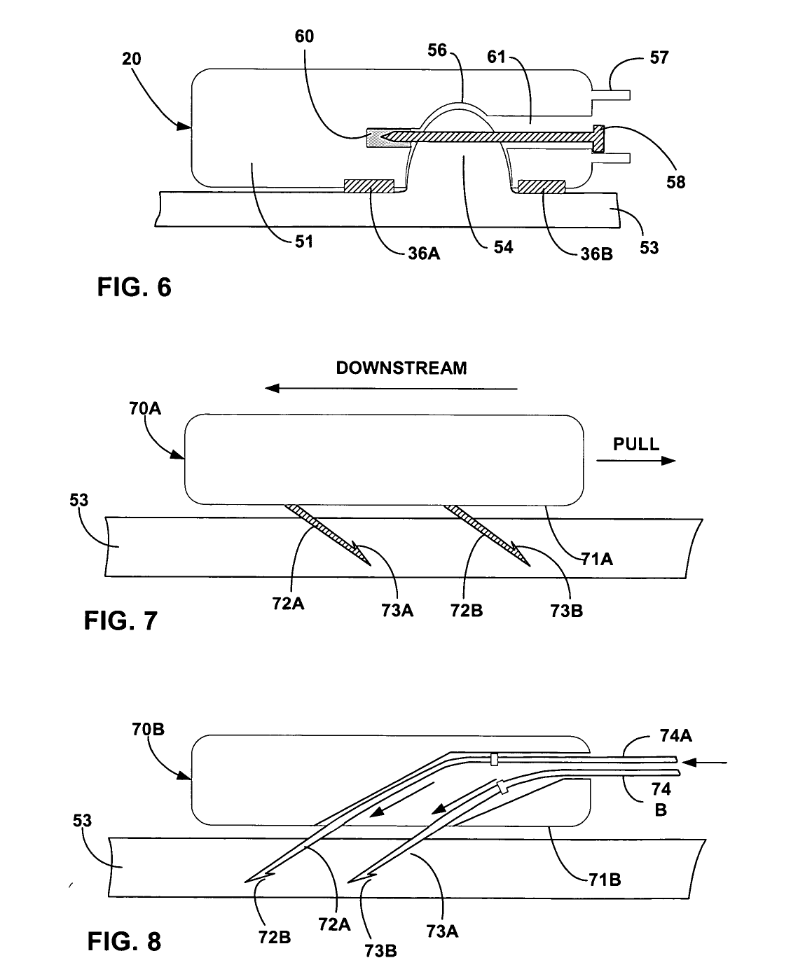

FIG. 6 is a cross-sectional side view of stimulation device 20 of FIG. 5 following removal of an endoscopic delivery device 40. As shown in FIG. 5, pin 58 holds device 20 securely in place relative to mucosal lining 53. At the same time, electrodes 36A, 36B are placed in contact with mucosal lining 53 to thereby deliver the electrical stimulation waveform to the target location. Electrodes 36A, 36B may operate as anode and cathode, respectively, for delivery of electrical stimulation. Electrodes 36A, 36B may be mounted to device housing 51 so that the electrodes are exposed to body tissue. For example, electrodes 36A, 36B may be in the form of conductive pads on one or both sides of vacuum cavity 56, or bands or rings that encircle the device housing on one or both sides of the vacuum cavity.

In other embodiments, tissue securing pin 58 may itself form an electrode, e.g., the cathode. In this case, one or more electrodes 36A, 36B may serve to create a common anode with tissue securing pin 58 forming the cathode. Bushing 60 may be electrically conductive and form part of an electrical conduction path between tissue securing pin 58 and the pulse generator housed within device housing 51. As tissue 54 captured within vacuum cavity 56 deteriorates, however, electrical conductivity between pin 58 and mucosal lining 53 may decrease. Therefore, it may be desirable to use electrodes 36A, 36B as anode and cathode in some applications for longer term delivery of electrical stimulation.

If a fixation structure that penetrates mucosal lining 53, such as pin 58, also serves as an electrode, it may be desirable to coat the surface of the fixation structure. For example, the fixation structure can be coated with a porous platinized structure to reduce polarization and/or an anti-inflammatory agent that inhibits inflammation that can negatively affect the ability to efficiently deliver electrical stimulation. The anti-inflammatory agents can be embedded into a monolithic controlled release device (MCRD) carried by the fixation structure. Such anti-inflammatory agents include steroids, anti-bacterial agents, baclofen, dexamethasone sodium phosphate and beclomethasone phosphate.

FIG. 7 is a side view of another stimulation device 70A within the gastrointestinal tract with a fixation structure using a pair of barbed hooks 72A, 72B to penetrate tissue within mucosal lining 53. Hooks 72A, 72B may be sized to limit the depth of penetration as described above, yet securely attach stimulation device 70A to the mucosal lining 53. Stimulation device 70A may have a capsule-like device housing 71 A, and may generally conform to stimulation device 20 of FIGS. 4-6. In the embodiment of FIG. 7, however, barbed hooks 72A, 72B function as the fixation structure and also form an anode and cathode for delivery of stimulation energy. A physician may deliver stimulation device 70 using an endoscopic device similar to delivery device 40 of FIGS. 3-6.

As an example, hooks 72A, 72B and associated barbs 73A, 73B may be angled upstream within the esophagus, as shown in FIG. 7, so that device housing 71 A can be maneuvered downstream without snagging the mucosal lining 53. Upon reaching the target location, e.g., within esophagus 14 or stomach 16, the physician may pull back on delivery device 20 to maneuver device housing 70A upstream and thereby snag and penetrate the mucosal lining 53 with hooks 72A, 72B.

Upon penetration of mucosal lining 53, hooks 72A, 72B secure stimulation device 70A in place at the target location, and the physician withdraws endoscopic delivery device 40. Stimulation device 70 then delivers electrical stimulation via hooks 72A, 72B, which are formed from electrical conductive material and form an anode and cathode, respectively. Although hooks 72A, 72B are described as serving as both the fixation structure and electrodes, in some embodiments, dedicated electrodes may be provided in addition to hooks 72A, 72B. In this case, hooks 72A, 72B may serve only for attachment, while electrodes are mounted to device housing 71A for contact with mucosal lining 53.

FIG. 8 is a side view of a stimulation device 70B within the gastrointestinal tract with an alternative fixation structure using a pair of barbed hooks 72A, 72B. In the example of FIG. 8, a physician actuates elongated translation members 74A, 74B via endoscopic delivery device 40 to push hooks 72A, 72B and extend them outward from device housing 71B to penetrate tissue within mucosal lining 53. During delivery to a target location, hooks 72A, 72B are withdrawn within device housing 71B. When device 70B arrives at the target location, however, the physician moves translation members 74A, 74B forward to extend hooks 72A, 72B. Translation members 74A, 74B may take the form of flexible push rods that force hooks 72A, 72B outward, but are then withdrawn from device housing 71B and removed from the body of patient 12 via delivery device 40.

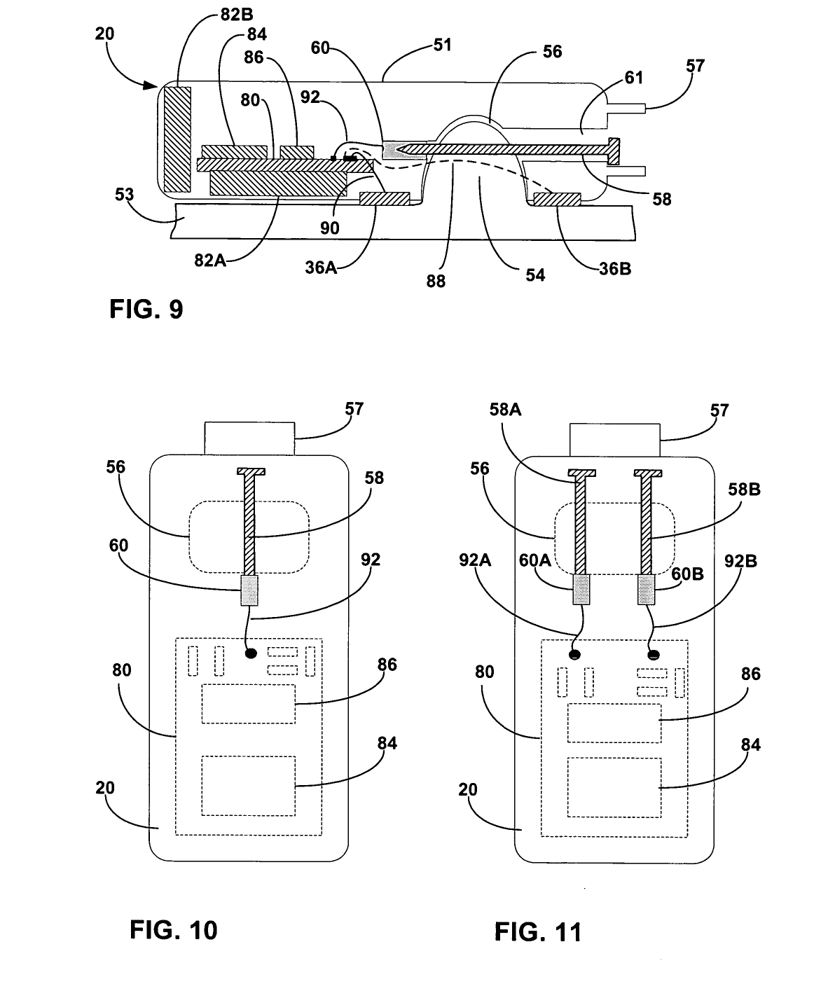

FIG. 9 is a cross-sectional side view illustrating exemplary arrangement of internal components of the stimulation device 20 shown in FIG. 4. FIG. 10 is a plan view of stimulation device 20 of FIG. 9. As shown in FIGS. 9 and 10, capsule-like device housing 51 contains a circuit board 80 with one or more integrated circuit devices 84, 86 and other electronics and associated electrical circuitry suitable for generating an electrical stimulation waveform. Various components of stimulation device 20, such as processor 26, memory 28, telemetry module 32, and pulse generator 34 (FIG. 2), may be mounted on circuit board 80. A battery or other power source also may be mounted on or proximate to circuit board 80. As illustrated in FIG. 9, a disk-shaped battery may be oriented in a variety of ways, such as substantially parallel to the gastrointestinal wall (82A) or substantially perpendicular to the gastrointestinal wall (82B). In the case of battery 82B, the disk-shaped battery may be substantially coaxial with a longitudinal axis of capsule-shaped housing 51, and may better fit the circular cross-section of the cylindrical housing.

As shown in FIG. 9, electrodes 36A, 36B may be coupled to terminals on circuit board 80 via wires 88, 90, respectively. If pin 58 forms an electrode, it also may be coupled to a terminal on circuit board 80, e.g., via a wire 92 coupled to conductive bushing 60. Wires 88, 90, 92 convey stimulation energy from pulse generator 34 to electrodes 36A, 36B, and optionally pin 58. In general, all components of stimulation device 20 are mounted within or to device housing 51. Therefore, there is no need for leads or other components to extend outside the body of patient 12. Instead, the entire stimulation device 20 is self-contained and resides within the gastrointestinal tract.

FIG. 11 is a plan view of an alternative stimulation device with a single vacuum cavity 56 and a pair of tissue securing pins 58A, 58B. Alternatively, each pin 58A, 58B may extend through a separate vacuum cavity. Pins 58A, 58B may form an anode and cathode, respectively, for delivery of stimulation energy to a portion 54 of mucosal lining tissue captured in vacuum cavity 56. In this case, stimulation current flows from one pin to the other. Pins 58A, 58B are coupled to terminals on circuit board 80 via wires 92A, 92B and conductive bushings 60A, 60B, respectively. In the example of FIG. 11, pins 58A, 58B may permit electrodes 36A, 36B to be eliminated.

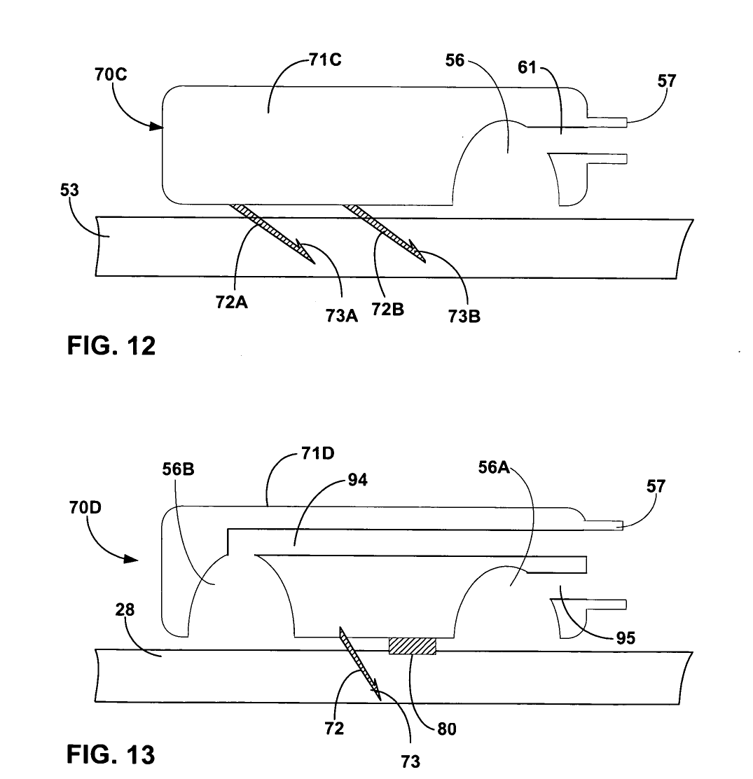

FIG. 12 is a cross-sectional side view of a stimulation device 70C with a fixation structure that combines barbed hooks 72A, 72B with a vacuum cavity 56 and vacuum port 61. Stimulation device 70C generally conforms to device 70A of FIG. 7, but further includes vacuum cavity 56 to draw mucosal lining 53 toward device 70A and thereby stabilize device housing 71 C against the mucosal lining during attachment of hooks 72A, 72B. In some embodiments, vacuum pressure may aid in driving hooks 72A, 72B into mucosal lining 53. Upon release of vacuum pressure, hooks 72A, 72B serve to secure stimulation device 70C to mucosal lining 53. Hooks 72A, 72B may be formed of conductive material to serve as electrodes, or separate electrodes may be mounted to device housing 71 C.

FIG. 13 is a cross-sectional side view of a stimulation device 70D with a fixation structure that combines barbed hook 72 with a pair of vacuum cavities 56A, 56B. Vacuum pressure applied to vacuum cavities 56A, 56B via vacuum ports 94, 95, respectively, draws mucosal lining 53 toward device 76C to thereby stabilize device body 71D against the mucosal lining, or aid in driving hook 72 into the mucosal lining. Upon release of vacuum pressure, hook 72 serves to secure stimulation device 70D to mucosal lining 53. Hook 72 may be formed of conductive material to serve as an electrode, e.g., in combination with electrode 80 mounted to device housing 71D. Alternatively, separate electrodes may be mounted to device housing 71D. In some embodiments, hook 72 may be extended from device housing 71D by actuating a translating member.

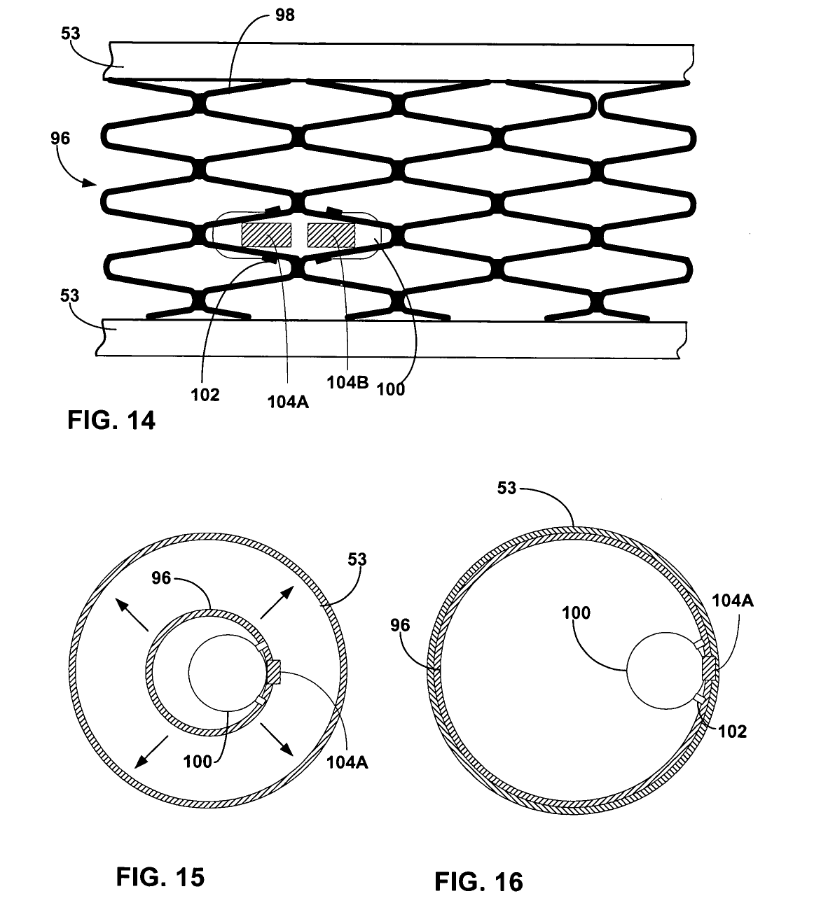

FIG. 14 is a side view of a stimulation device 100 with a fixation structure in the form of an expandable frame 96. FIGS. 15 and 16 are cross-sectional views of device 100 and expandable frame 96 in an unexpanded state and expanded state, respectively, within a body lumen. As shown in FIGS. 14-16, capsule-like stimulation device 100 is attached to a portion of a wire grid 98 forming expandable frame 96. Stimulation device 100 may be welded, adhesively bonded, or crimped to a one or more coupling points 102 on expandable frame 96.

Wire grid 98 may take the form of a grid, network, or mesh of elastic wires that form a substantially cylindrical frame 96, similar to a conventional stent useful in restoring blood vessel patency. Examples of suitable materials for fabrication of wire grid 98 include stainless steel, titanium, nitinol, and polymeric filament, which can be absorbable or nonabsorbable in vivo, as described in the reference Kilcoyne patents. Expandable frame 96 may be intrinsically elastic such that it is self-expandable upon release from a restraint provided by an endoscopic delivery device. Alternatively, in some embodiments, a balloon or other actuation mechanism may be used to actively expand frame 96 to a desired diameter.

In each case, as shown in FIGS. 15 and 16, expandable frame 96 extends radially outward to engage the wall of a body lumen, such as the esophagus or small intestine, and thereby place stimulation device 100 in contact with the lumen wall. In particular, upon expansion of frame 96, one or more electrodes 104A, 104B are placed in contact with the mucosal lining of the body lumen, permitting delivery of an electrical stimulation waveform.

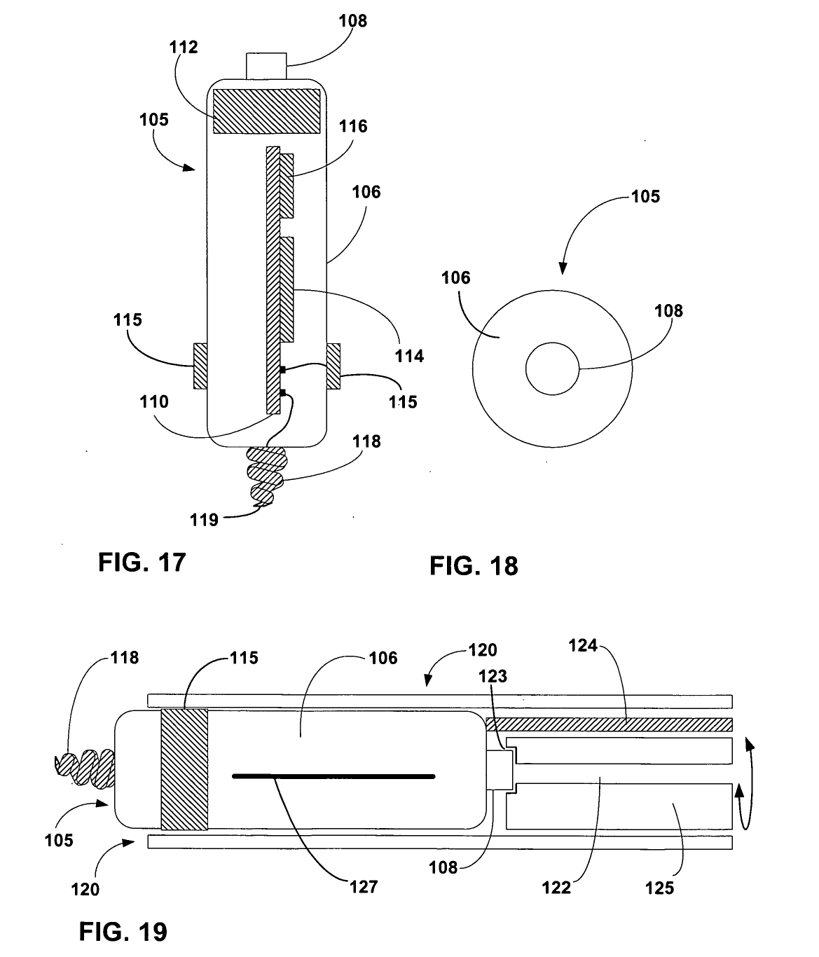

FIG. 17 is cross-sectional side view of another stimulation device 105 with a capsule-like device housing 106. FIG. 18 is a top view of stimulation device 105 of FIG. 17. As shown in FIGS. 17 and 18, stimulation device housing 106 includes a raised feature 108, an internal circuit board 110 carrying components 114, 116 and coupled to a battery 112, a ring-like electrode 115, and a screw-like extension 118 extending from an end of the housing opposite the raised feature.

Ring-like electrode 115 may extend about the entire periphery or a portion of the periphery of stimulation device housing 106. In the illustrated embodiment, screw-like extension may be formed from an electrically conductive material, in which case ring-like electrode 115 and screw-like extension 118 may serve as an anode and cathode, respectively, for stimulation device 105. In other embodiments, two or more ring-like electrodes, similar to electrode 115, may be provided to serve as cathode and anode for delivery of stimulation energy.

Stimulation device 105 is capable of delivery via an endoscopic delivery device, but includes an axial fixation structure rather than a lateral fixation structure. In particular, screw-like extension 118 extends coaxially with the longitudinal axis of stimulation device 105. During placement of stimulation device 105, screw-like extension 118 extends distally from the delivery device. Helical screw-like extension 118 may include one or more helical coil turns terminating in sharpened tip 119.

FIG. 19 is a cross-sectional side view of stimulation device 105 of FIG. 17, illustrating delivery via an endoscopic delivery device 120. As shown in FIG. 19, device housing 106 is disposed at a distal end 121 of delivery device 120. Raised feature 108 engages a recess 123 within a working member 125 of delivery device 120. Recess 123 is coupled to a vacuum port 122. A physician applies vacuum pressure to raised feature 108 via recess 123 and vacuum line 122 to hold device housing 106 in place during delivery to the target location within the gastrointestinal tract.

When distal end 121 of delivery device 120 reaches a target location, the physician rotates working member 125 to rotate stimulation device 105 and thereby screw extension 118 into the target site. The physician then deactivates the vacuum pressure, and advances a translation member 124 to push stimulation device 105 out of delivery device 120 to ensure separation, and withdraws delivery device 120. Device housing 106 may include one of more longitudinal markings 127 to permit a physician to see, with endoscopic visualization, to what extent stimulation device 105 has been rotated during screw-in insertion into tissue. Alternatively, the markings 127 may be radio-opaque to permit external visualization using radiography or fluoroscopy.

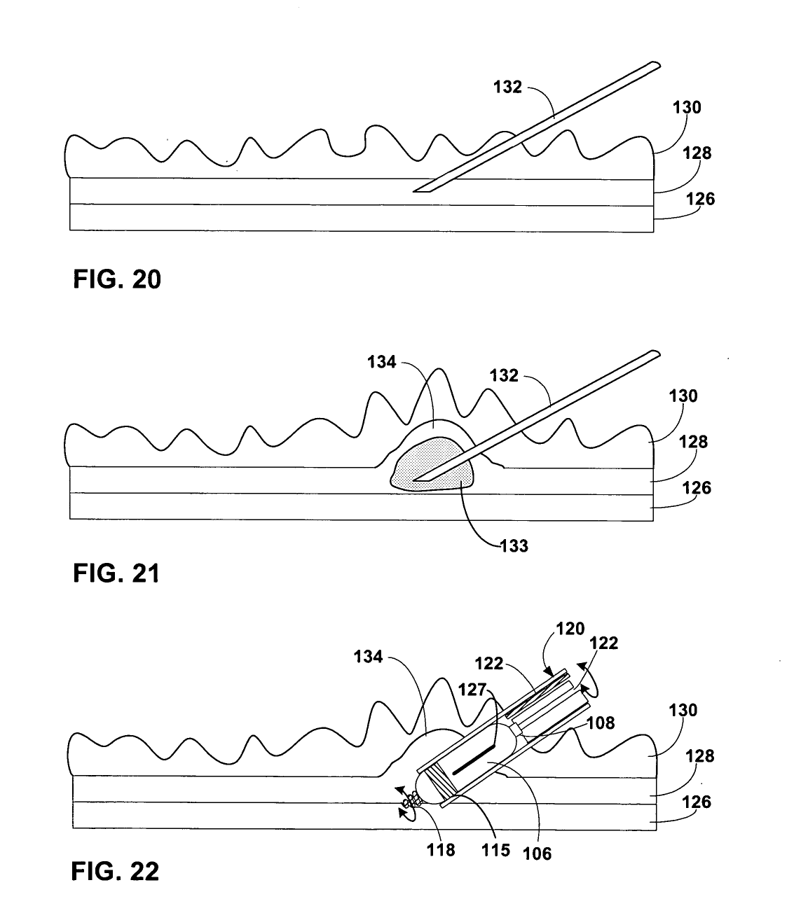

FIG. 20 is a schematic diagram illustrating insertion of a stylet 132 into the mucosal lining of the stomach as part of an exemplary procedure for implantation of stimulation device 105 of FIGS. 17-19. As shown in FIG. 20, stylet 132 is endoscopically guided to a target location within the lumen of the stomach. At the target location, the stomach lining includes muscle layer 126, submucosal layer 128 and mucosal layer 130. Stylet 132 penetrates submucosal layer 128.

FIG. 21 is a schematic diagram illustrating introduction of fluid 133, such as saline, through stylet 132 to create an expanded implant pocket 134. To insert stimulation device 105 into sub-mucosal layer 128 so that the screw-like extension 118 makes electrical contact with muscle tissue and associated sub-mucosal plexus or myenteric plexus, it is necessary to first create pocket 134 in the sub-mucosal layer. The volume of fluid 133 introduced by stylet 132 expands submucosal layer 128 to create a pocket-like protrusion. The introduction of saline into sub-mucosal layer 128 results in a sort of a saline “blister.”

Upon creation of the implant pocket 134, the physician withdraws stylet 132 and makes a small incision in the blister with a small endoscopic cutting instrument. The physician then introduces endoscopic delivery device 120 through the incision opening in the blister to deliver stimulation device 105, as shown in FIG. 22. When the screw-like extension makes contact with muscle layer 126 of the stomach, the physician screws the capsule-like stimulation device 105 into the muscle layer, e.g., with one turn of the device.

When translation member 124 is advanced to force stimulation device housing 106 out of delivery device 120, screw-like extension 118 is lodged in the muscle layer tissue. Then, the physician deactivates vacuum pressure, and withdraws endoscopic delivery device 120 slightly so that the proximal end of the stimulation device 105 is fully visible. The physician then places the capsule-like housing 106 placed fully within pocket 134, and closes the pocket, e.g., with sutures or clips applied endoscopically. Then, the physician withdraws delivery device 120 from patient 12, leaving stimulation device 105 in place within the stomach lining. In this manner, a self-contained, capsule-like stimulation device 105 is securely implanted within the patient, and operates without the need for trans-nasal or trans-oral leads that could otherwise cause discomfort for the patient or result in dislodgement of electrodes.

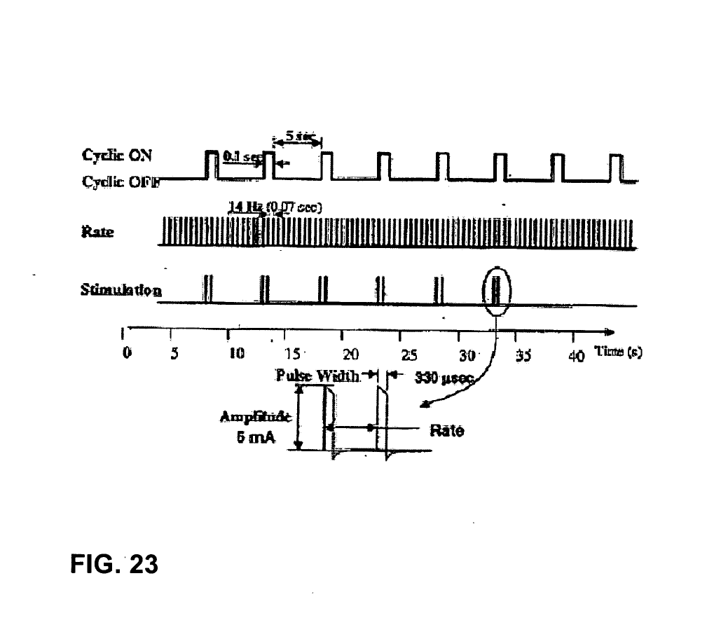

FIG. 23 is a timing diagram illustrating various parameters of an electrical stimulation waveform for gastrointestinal stimulation. In general, a stimulation device in accordance with the invention may deliver any of a variety of electrical stimulation waveforms with parameters selected to alleviate undesirable symptoms associated with a given gastrointestinal disorder such as symptoms of nausea, vomiting or gastric discomfort. In some embodiments, the parameters may be selected not only to suppress symptoms, but also to alleviate the cause of the symptoms. As an example, the parameters may be selected to treat gastroparesis by providing a stimulation waveform that is effective in restoring gastric motility. An exemplary electrical stimulation waveform can be characterized by a set of signal parameters including amplitude, frequency, pulse width, and duty cycle. An additional parameter is the duration for which the electrical stimulation waveform is applied.

A suitable electrical stimulation waveform for alleviating symptoms of nausea and vomiting may have an amplitude in the range of approximately 0.1 to 10 mA, and preferably approximately 5 mA. In addition, the electrical stimulation waveform may have a frequency of approximately 10 to 250 Hz, and preferably approximately 14 Hz as shown in FIG. 23, a pulse width of approximately 100 to 100 microseconds, and preferably approximately 330 microseconds as shown in FIG. 23, and a duty cycle with an on period of approximately 0.1 to 0.5 seconds, and preferably approximately 0.1 seconds as shown in FIG. 23, and an off period of approximately 1 to 10 seconds, and preferably approximately 5 seconds, as shown in FIG. 23. The above parameter settings have been observed to provide effective relief of symptoms such as nausea and vomiting in many patients. The electrical stimulation waveform may be applied for a duration of several minutes, e.g., 5 to 30 minutes, and then turned off and reapplied periodically when symptoms recur. Alternatively, in some embodiments, the electrical stimulation waveform may be applied continuously.

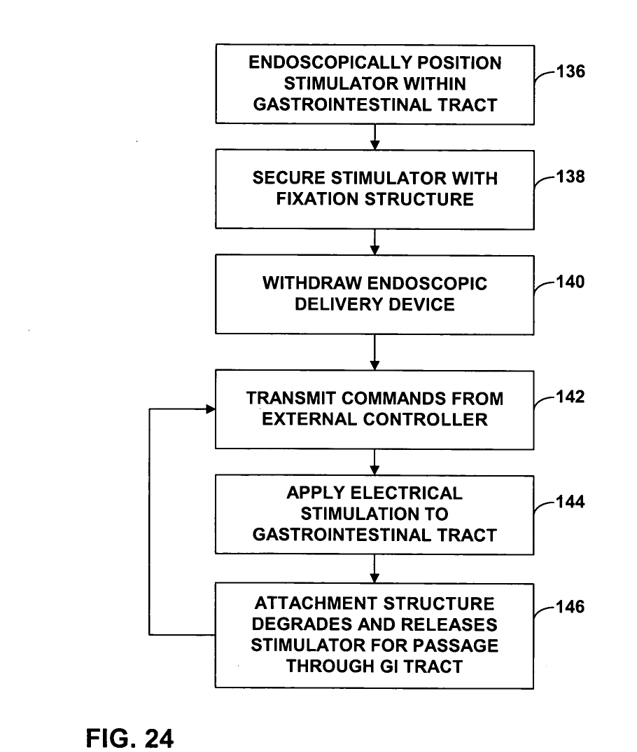

FIG. 24 is a flow diagram illustrating implantation and operation of a gastrointestinal electrical stimulator. As shown in FIG. 24, the physician positions the capsule-like stimulator at a target location within the gastrointestinal tract with an endoscopic delivery device (136) and then secures the stimulator to tissue at the target location using a fixation structure carried by the stimulator (138). Upon withdrawing the endoscopic delivery device from the patient (140), the physician may transmit one or more commands to the implanted stimulation device using an external controller to activate the stimulation device (142). Alternatively, the stimulation device may be self-activating upon deployment from the endoscopic delivery device. If an external controller is provided, in some embodiments, it also may be used to adjust stimulation parameter settings.

Upon activation, the capsule-like stimulation device applies electrical stimulation waveform to the target location within the gastrointestinal tract (144). The stimulation device continues to operate until battery resources are exhausted or, in some embodiments in which the fixation structure is made from a degradable material, the fixation structure degrades and releases the stimulator from the target tissue to permit the stimulator to pass through the gastrointestinal tract (146). As a further alternative, the stimulator may release from the tissue as the tissue deteriorates and sloughs away, permitting the stimulation device to pass through the gastrointestinal tract.

The preceding specific embodiments are illustrative of the practice of the invention. It is to be understood, therefore, that other expedients known to those skilled in the art or disclosed herein may be employed without departing from the invention or the scope of the claims. For example, the invention is not limited to deployment of a stimulation device at a particular location within the gastrointestinal tract. In various embodiments, a stimulation device may be located anywhere within the gastrointestinal tract. For example, the stimulation device may be affixed along or to any of the other structures and organ walls along the gastrointestinal tract, including the colon, small intestine, stomach, or the esophagus.

In addition, the invention is not limited to application for any particular disorder, condition or affliction. As examples, the invention may be applicable to treatment of symptoms secondary to a variety of conditions, such as nausea or vomiting secondary to gastroparesis, functional dyspepsia, chemotherapy, post-operative ileus, or even pregnancy. Also, the invention may be applicable not only to treat particular short-term or mid-term symptoms, but also for trial stimulation to evaluate the efficacy of stimulation for a variety of treatments such as more long-term treatment of gastroparesis, obesity, irritable bowel syndrome, functional dyspepsia, and gastroesophageal reflux disease, to name a few.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Many embodiments of the invention have been described. Various modifications may be made without departing from the scope of the claims. These and other embodiments are within the scope of the following claims.

1. A device for electrical stimulation of a gastrointestinal tract of a patient, the device comprising:
a device housing sized for introduction into a gastrointestinal tract;
an electrical pulse generator, mounted within the device housing, to generate an electrical stimulation waveform;
one or more electrodes electrically coupled to the electrical pulse generator and mounted to the device housing to deliver the electrical stimulation waveform to the gastrointestinal tract; and
a fixation structure to attach the device housing to a surface within the gastrointestinal tract.

2. The device of claim 1, wherein the device housing has a substantially cylindrical capsule-like shape.

3. The device of claim 1, wherein the fixation structure includes a cavity formed in the device housing and a pin to penetrate gastrointestinal tissue within the cavity.

4. The device of claim 3, wherein the cavity includes a vacuum port for application of vacuum pressure to draw the tissue into the cavity.

5. The device of claim 3, wherein the pin forms one of the electrodes.

6. The device of claim 1, wherein the fixation structure includes two or more cavities, and vacuum ports for application of vacuum pressure to draw tissue into the cavities.

7. The device of claim 1, wherein the fixation structure includes one or more barbed hooks that extend from the device housing to penetrate gastrointestinal tissue.

8. The device of claim 7, wherein the barbed hooks form at least one of the electrodes.

9. The device of claim 1, wherein the fixation structure includes a screw-like extension that extends from the device housing to penetrate gastrointestinal tissue.

10. The device of claim 9 wherein the screw-like extension forms one of the electrodes.

11. The device of claim 9, wherein the screw-like extension extends from a distal end of the device housing.

12. The device of claim 1, wherein the fixation structure includes an expandable frame that is expandable radially outward to contact a lumen wall within the gastrointestinal tract, and the device housing is mounted to the expandable frame.

13. The device of claim 1, further comprising a power source mounted within the device housing, and the power source is coupled to the pulse generator.

14. The device of claim 13, wherein the power source includes a substantially disc-shaped battery.

15. The device of claim 1, wherein the fixation structure forms one of the electrodes.

16. The device of claim 1, wherein the electrodes include a first electrode and a second electrode mounted on an exterior surface of the device housing for electrical contact with tissue within the gastrointestinal tract.

17. The device of claim 1, wherein the device housing has a substantially cylindrical capsule-like shape, and at least one of the electrodes includes an electrode ring that extends about a circumference of the device housing.

18. The device of claim 1, wherein the device housing has maximum length of less than approximately 10 mm and a maximum width of less than approximately 5 mm.

19. The device of claim 1, wherein the fixation structure is degradable to permit detachment of the device housing from the tissue and passage of the device through the gastrointestinal tract.

20. The device of claim 1, wherein the pulse generator generates a stimulation waveform selected to suppress one or more symptoms including at least one of nausea and vomiting.

21. The device of claim 1, wherein the pulse generator generates a stimulation waveform selected to suppress one or more symptoms secondary to at least one of gastroparesis, functional dyspepsia, chemotherapy, post-operative ileus, and pregnancy.

22. The device of claim 1, wherein the pulse generator generates a stimulation waveform with an amplitude in a range of approximately 0.1 mA to 10 mA, a frequency in a range of approximately 10 Hz to 250 Hz, a pulse width in a range of approximately 100 microseconds to 1000 microseconds, an on duty cycle in a range of approximately 0.1 seconds to 0.5 seconds, and an off duty cycle in a range of approximately 1 second to 10 seconds.

23. The device of claim 1, wherein the pulse generator generates a stimulation waveform with an amplitude of approximately 5 mA, a frequency of approximately 14 Hz, a pulse width of approximately 330 microseconds, an on duty cycle of approximately 0.1 seconds, and an off duty cycle of approximately 5 seconds.

24. The device of claim 1, wherein the device includes no leads that extend outside of a body of a patient upon placement of the device within the gastrointestinal tract.

25. A device for electrical stimulation of a gastrointestinal tract of a patient, the device comprising:
a device housing sized for introduction into a gastrointestinal tract;
means, mounted within the device housing, for generating an electrical stimulation waveform selected to suppress one of more symptoms of gastroparesis;
one or more electrodes electrically coupled to the means for generating an electrical stimulation waveform and mounted to the device housing to deliver the electrical stimulation waveform to the gastrointestinal tract; and
means for attaching the device housing to a surface within the gastrointestinal tract.

26. The device of claim 25, wherein the means for attaching includes a cavity formed in the device housing and a pin to penetrate gastrointestinal tissue within the cavity, the cavity including a vacuum port for application of vacuum pressure to draw the tissue into the cavity.

27. The device of claim 25, wherein the means for attaching includes one or more barbed hooks that extend from the device housing to penetrate gastrointestinal tissue.

28. The device of claim 25, wherein the means for attaching includes a screw-like extension that extends from the device housing to penetrate gastrointestinal tissue.

29. The device of claim 25, wherein the means for attaching includes an expandable frame that is expandable radially outward to contact a lumen wall within the gastrointestinal tract, and the device housing is mounted to the expandable frame.

30. The device of claim 25, further comprising means for supplying power to power the means for generating an electrical stimulation waveform, wherein the power supplying means is mounted within the device housing.

31. The device of claim 30, wherein the power supplying means includes a substantially disc-shaped battery.

32. The device of claim 25, wherein the means for attaching forms one of the electrodes.

33. The device of claim 25, wherein the device housing has maximum length of less than approximately 10 mm and a maximum width of less than approximately 5 mm.

34. The device of claim 25, wherein the means for attaching is degradable to permit detachment of the device housing from the tissue and passage of the device through the gastrointestinal tract.

35. The device of claim 25, wherein the means for generating the stimulation waveform generates a stimulation waveform selected to suppress one or more symptoms including at least one of nausea and vomiting.

36. The device of claim 25, wherein the means for generating the stimulation waveform generates a stimulation waveform selected to suppress one or more symptoms secondary to at least one of gastroparesis, functional dyspepsia, chemotherapy, post-operative ileus, and pregnancy.

37. The device of claim 25, wherein the means for generating the stimulation waveform generates a stimulation waveform with an amplitude in a range of approximately 0.1 mA to 10 mA, a frequency in a range of approximately 10 Hz to 250 Hz, a pulse width in a range of approximately 100 microseconds to 1000 microseconds, an on duty cycle in a range of approximately 0.1 seconds to 0.5 seconds, and an off duty cycle in a range of approximately 1 second to 10 seconds.

38. The device of claim 25, wherein the means for generating the stimulation waveform generates a stimulation waveform with an amplitude of approximately 5 mA, a frequency of approximately 14 Hz, a pulse width of approximately 330 microseconds, an on duty cycle of approximately 0.1 seconds, and an off duty cycle of approximately 5 seconds.

39. A method for electrical stimulation of a gastrointestinal tract of a patient, the method comprising:
placing an electrical stimulation device at a target location with the gastrointestinal tract;
attaching a device housing to tissue at the target location with a fixation structure mounted to the device housing;
generating an electrical stimulation waveform with an electrical pulse generator mounted within the device housing; and
delivering the electrical stimulation waveform to the gastrointestinal tract with electrodes coupled to the pulse generator and mounted to the device housing.

40. The method of claim 39, wherein placing the stimulation device includes endoscopically placing the stimulation device with an endoscopic delivery device introduced into an esophagus of the patient.

41. The method of claim 39, wherein attaching the device housing includes applying vacuum pressure to a cavity within the device housing to draw tissue into the cavity, and advancing a pin to penetrate the tissue within the cavity.

42. The method of claim 41, wherein the pin forms one of the electrodes.

43. The method of claim 39, wherein attaching the device housing includes penetrating gastrointestinal tissue with one or more barbed hooks.

44. The method of claim 43, wherein the barbed hooks form at least one of the electrodes.

45. The method of claim 39, wherein attaching the device housing includes penetrating gastrointestinal tissue with a screw-like extension that extends from the device housing to penetrate gastrointestinal tissue.

46. The method of claim 45, wherein the screw-like extension forms one of the electrodes.

47. The method of claim 39, wherein attaching the device housing includes expanding an expandable frame radially outward to contact a lumen wall within the gastrointestinal tract, the device housing being mounted to the expandable frame.

48. The method of claim 39, further comprising powering the pulse generator with a power source mounted within the device housing.

49. The method of claim 48, wherein the power source includes a substantially disc-shaped battery.

50. The method of claim 39, wherein the fixation structure is degradable to permit detachment of the device housing from the tissue and passage of the device through the gastrointestinal tract.

51. The method of claim 39, wherein the stimulation waveform has parameters selected to suppress one or more symptoms including at least one of nausea and vomiting.

52. The method of claim 39, wherein the stimulation waveform has parameters selected to suppress one or more symptoms secondary to at least one of gastroparesis, functional dyspepsia, chemotherapy, post-operative ileus, and pregnancy.

53. The method of claim 39, wherein the stimulation waveform has an amplitude in a range of approximately 0.1 mA to 10 mA, a frequency in a range of approximately 10 Hz to 250 Hz, a pulse width in a range of approximately 100 microseconds to 1000 microseconds, an on duty cycle in a range of approximately 0.1 seconds to 0.5 seconds, and an off duty cycle in a range of approximately 1 second to 10 seconds.

54. The method of claim 39, wherein the stimulation waveform has an amplitude of approximately 5 mA, a frequency of approximately 14 Hz, a pulse width of approximately 330 microseconds, an on duty cycle of approximately 0.1 seconds, and an off duty cycle of approximately 5 seconds.

55. The method of claim 39, further comprising delivering the electrical stimulation waveform to treat symptoms of nausea and vomiting following chemotherapy.

56. The method of claim 39, further comprising delivering the electrical stimulation waveform to treat symptoms of nausea and vomiting following surgery.
